(19) United States
(12) Patent Application Publication
(10) Pub. No.: US 2010/0273241 A1
(43) Pub. Date: Oct. 28, 2010

(54) METHOD FOR MANUFACTURING RED MOLD DIOSCOREA

(75) Inventors: Tzu-Ming Pan, Sijhih City (TW); Chun-Lin Lee, Fongshan City (TW)

Correspondence Address: ROGER H. CHU, 19499 ERIC DRIVE, SARATOGA, CA, 95070, US

(73) Assignee: SUNWAY BIOTECH CO., LTD., Taipei City (TW)

(21) Appl. No.: 12/690,379

(22) Filed: Jan. 20, 2010

(30) Foreign Application Priority Data
Apr. 24, 2009 (TW) 098113815

(51) Int. Cl. C12N 1/16

(52) U.S. Cl. 435/255.1

(57) ABSTRACT

The present invention discloses a method for manufacturing red mold dioscorea, and the method comprises the following steps: adding some water with an appropriate ratio to fresh dioscorea or dried dioscorea; sterilizing the dioscorea; after the dioscorea being cooled down, inoculating the dioscorea with Monascus species; cultivating the dioscorea with an appropriate temperature, an appropriate humidity and an appropriate shacking frequency for an appropriate time period; and drying the cultivated red mold dioscorea with an appropriate water content.

### CROSS-REFERENCE TO RELATED APPLICATION

This application claims foreign priority from a Taiwan Patent Application, Ser. No. 098113815, filed on Apr. 24, 2009.

### BACKGROUND OF THE INVENTION

1. Field of the Invention

The present invention relates to a method for manufacturing red mold dioscorea, and more particularly relates to a method for manufacturing red mold dioscorea with a solid state cultivation method or a liquid state cultivation method.

2. Description of the Prior Art

Hypertension caused by hypercholesterol is a popular civilization disease in recent years. Following the advancement of medical science, populations of middle-aged and geriatric people are increasing. It is difficult to eliminate superoxide free radicals in middle-aged and geriatric people result from the lower metabolic mechanism, and the middle-aged and geriatric diseases include cancer and cardiopathy are prone to occur. Cardiovascular diseases arise from hypercholesterolemia include apoplexy, coronary atherosclerotic cardiopathy and hypertension. In pursue of the healthy diet in life, health food nowadays is very popular. Red mold products possess both effects of inhibition of cholesterol synthesis and blood pressure lowering and become one of the popular health food.

Numerous researches in recent years prove that red mold rice have significant lipid-lowering effect on hyperlipidemia hamsters, thus red mold rice have potential to be an ingredient in cholesterol-lowering drugs and improve this kind of disease.

The above effects are result from that red mold rice can produce several kinds of secondary metabolite, such as cholesterol-synthesis inhibitor, blood pressure lowering material, anti-cancer material and so forth. Some previous researches about secondary metabolite of red mold yeast indicate that the yield and pattern of secondary metabolite may be directly or indirectly influenced by cultural environment and method.

In the past, red mold related products are all made from rice as the fermented substrate, but the yields of anti-inflammation material (monascin), anti-cancer material (ankaflavin) and cholesterol-lowering material (monacolin K) are not abundant in red mold rice. In view of the above problem, it is necessary to choose a fermented substrate with better effects and adjust the culture conditions to be optimum so as to elevate the effects of blood-lipid lowering, blood-pressure lowering and atherosclerosis prevention.

### SUMMARY OF THE INVENTION

In view of the above shortcomings of the prior art, the inventor of the present invention resorted to past experience, imagination, and creativity, performed experiments and researches repeatedly, and eventually devised the present invention—a method for manufacturing red mold dioscorea.

The first objective of the present invention is to provide a method for manufacturing red mold dioscorea, owing to the amounts of each kind of secondary metabolite produced by red mold yeast are variant under different cultural conditions, the manufacturing method and all the cultural conditions according to the present invention can promote the amount of formation of effective secondary metabolites, such as anti-inflammation material (monascin), anti-cancer material (ankaflavin) and cholesterol-lowering material (monacolin K), which can be used for compositions of drugs for lowering blood lipid, lowering blood pressure and preventing atherosclerosis.

The second objective of the present invention is to provide a method for manufacturing red mold dioscorea, owing to the amounts of secondary metabolites produced by red mold yeast are variant with different substrates, dioscorea is used for the substrate for culturing red mold yeast according to the present invention to promote the amount of formation of effective secondary metabolites, such as anti-inflammation material (monascin), anti-cancer material (ankaflavin) and cholesterol-lowering material (monacolin K), which can be used for compositions of drugs for lowering blood lipid, lowering blood pressure and preventing atherosclerosis.

Consequently, the present invention provides a method for manufacturing red mold dioscorea, the method is a solid state cultivation method and comprising the steps of: washing a fresh dioscorea clean and cutting the fresh dioscorea into pieces with a specific dimension; drying or sulfuring the pieces of the fresh dioscorea to a specific water content; adding a specific amount of water to the dried dioscorea to make the dried dioscorea and the water be a specific ratio and soaking the dried dioscorea for a specific time period; proceeding a sterilization process to the soaked dioscorea and then cooling the product down to a specific temperature; inoculating a Monascus spp. to the sterilized dioscorea; culturing the inoculated dioscorea under a specific cultural temperature and a specific cultural humidity for a specific cultural time period; proceeding an anaerobic treatment to the product of previous step for a specific treating time period; and drying the product of previous step to a specific water content so as to accomplish the manufacture of red mold dioscorea.

The present invention further provides another method for manufacturing red mold dioscorea, the method is a liquid state cultivation method and comprising the steps of: adding a specific amount of water to a raw material of a dioscorea to make the raw material of the dioscorea and the water be a specific ratio; proceeding a sterilization process to the raw material of the dioscorea and then cooling the product down to a specific temperature; inoculating a Monascus spp. to the sterilized dioscorea; culturing the inoculated dioscorea under a specific cultural temperature and a specific shaking frequency for a specific cultural time period; proceeding an anaerobic treatment to the product of previous step for a specific treating time period; treating the product of previous step with a centrifugation process; and drying the product of previous step to a specific water content so as to accomplish the manufacture of red mold dioscorea.

### BRIEF DESCRIPTION OF THE DRAWINGS

FIG. 1 is a flowchart of a method for manufacturing red mold dioscorea according to a first preferred embodiment of the present invention;

FIG. 2 is a flowchart of the method for manufacturing the red mold dioscorea according to a second preferred embodiment of the present invention;

FIG. 3 is a flowchart of the method for manufacturing the red mold dioscorea according to a third preferred embodiment of the present invention;

FIG. 4 is a flowchart of the method for manufacturing the red mold dioscorea according to a fourth preferred embodiment of the present invention;

FIG. 5 is a trend graph of yellow pigment and red pigment formation in the cultural processes of the red mold rice and red mold dioscorea; and

FIG. 6 is a trend graph of monacolin K formation in the cultural processes of the red mold rice and red mold dioscorea.

### DETAILED DESCRIPTION OF THE PREFERRED EMBODIMENTS

To achieve the foregoing objectives and effects, the inventors integrate red mold yeast with dioscorea and improve and amend the conditions of manufacturing methods, thus achieving the method for manufacturing red mold dioscorea of the present invention. Hereinafter, flowcharts of methods for manufacturing red mold dioscorea according to a first preferred embodiment, a second preferred embodiment, a third preferred embodiment, and a fourth preferred embodiment of the present invention will be described in detail for illustrating the method for manufacturing red mold dioscorea.

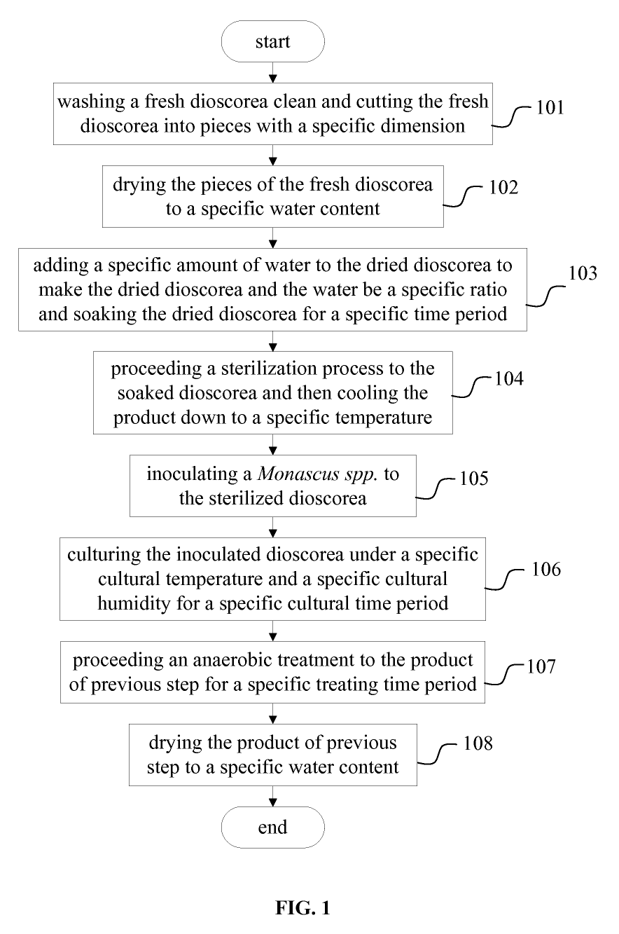

Referring to FIG. 1, which is a flowchart of a method for manufacturing red mold dioscorea according to the first preferred embodiment of the present invention. The method includes the following step (101) of washing a fresh dioscorea clean and cutting the fresh dioscorea into pieces with a specific dimension, wherein the specific dimension is 2˜20 mm, and the optimal dimension is 2 mm; step (102) of drying the pieces of the fresh dioscorea to a specific water content, wherein the specific water content is below 15%, and the optimal water content is 7%; step (103) of adding a specific amount of water to the dried dioscorea to make the dried dioscorea and the water be a specific ratio and soaking the dried dioscorea for a specific time period, wherein the specific ratio is 1:0.5%˜1:1.5%, the optimal ratio is 1:0.75%, the specific time period is 60 minutes, and the optimal time is 30 minutes; step (104) of proceeding a sterilization process to the soaked dioscorea and then cooling the product down to a specific temperature, wherein the sterilization process is a high temperature sterilization process, a temperature for the sterilization process is 121° C., and a time period for the sterilization process is 10˜60 minutes; step (105) of inoculating a Monascus spp. to the sterilized dioscorea; step (106) of culturing the inoculated dioscorea under a specific cultural temperature and a specific cultural humidity for a specific cultural time period, wherein the specific cultural temperature is 25˜37° C., the optimal cultural temperature is 30° C., the specific cultural humidity is 50˜80%, the optimal cultural humidity is 60%, the specific cultural time period is 8˜20 days, and the optimal cultural time is 10 days; step (107) of proceeding an anaerobic treatment to the product of previous step for a specific treating time period, wherein the specific treating time period is 3 days, and the optimal treating time period is 2 days; and step (108) of drying the product of previous step to a specific water content so as to accomplish the manufacture of red mold dioscorea, wherein the specific water content is below 15%, and the optimal water content is 6%.

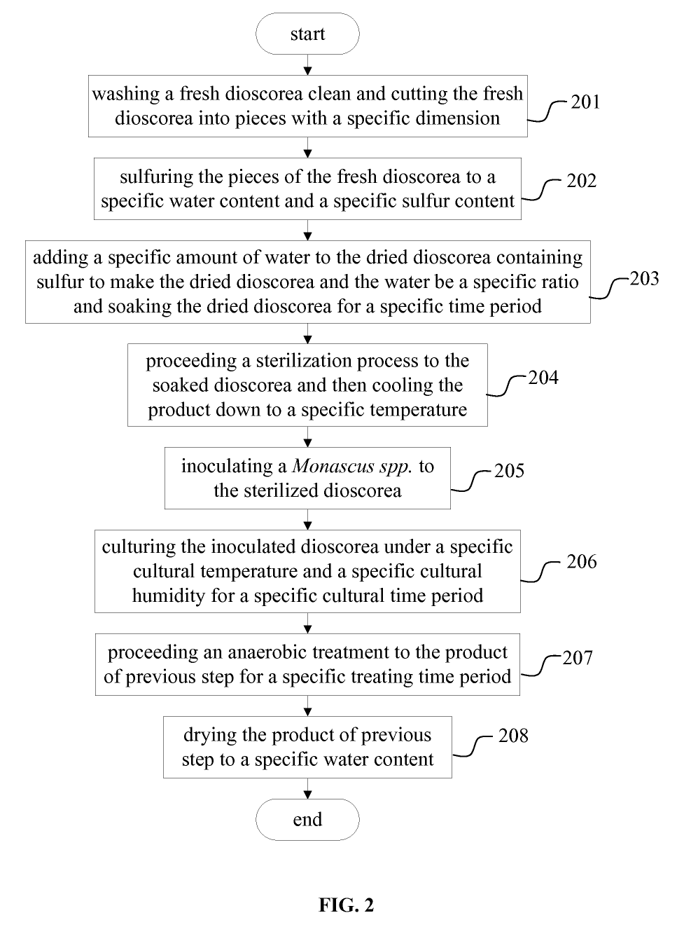

Referring to FIG. 2, which is a flowchart of a method for manufacturing red mold dioscorea according to the second preferred embodiment of the present invention. The steps (201) and (203)˜(208) of the second preferred embodiment are almost the same as the steps (101) and (103)˜(108) of the first preferred embodiment, but there are some differences between step (102) and step (202). The fresh dioscorea in step (102) of the first preferred embodiment is dried by a normal drying method and the purpose of the normal drying method is merely decreasing the water content of the fresh dioscorea. In stead of the drying method in step (102), the pieces of the fresh dioscorea in step (202) of the second preferred embodiment is dried by a sulfuring method to reach a specific water content and a specific sulfur content, wherein the specific water content is below 15%, the optimal water content is 7%, the specific sulfur content is below 160 ppm, and the optimal sulfur content is 120 ppm. The purpose of the sulfuring method is to make the dioscorea containing a trace of sulfur which can inhibit the growth of microorganisms and enhance the effect of preservation.

After adding the specific amount of water to the dried dioscorea in step (103) and (203) of the foregoing first and second preferred embodiment, the ratio of the dioscorea and the water is 1:0.5%˜1:1.5%, it is obvious that only a small amount of water is added. The dioscorea is kept in a solid form of a dioscorea substrate after being sterilized with high temperature, thus the two methods for culturing red mold yeast with the solid form of the dioscorea substrate are called solid state cultivation methods.

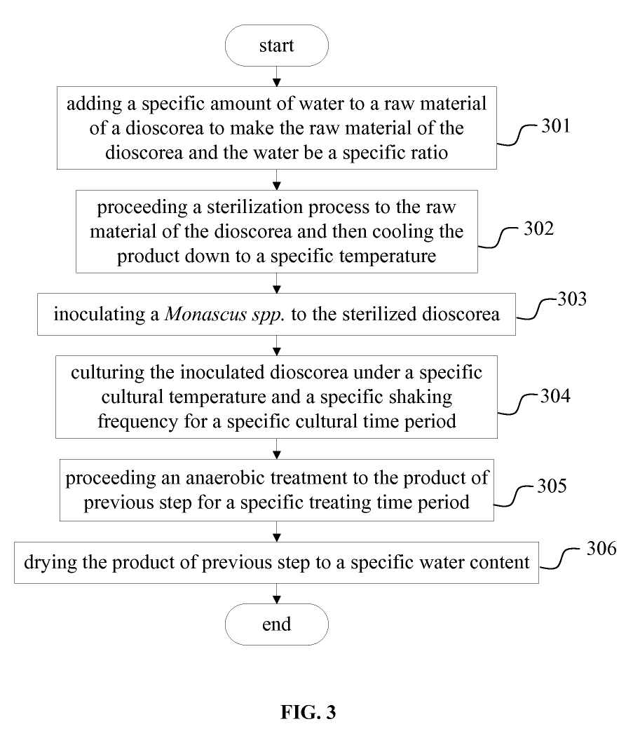

Referring to FIG. 3, which is a flowchart of a method for manufacturing red mold dioscorea according to the third preferred embodiment of the present invention. The method includes the following step (301) of adding a specific amount of water to a raw material of a dioscorea to make the raw material of the dioscorea and the water be a specific ratio, wherein the specific ratio is 1:10˜1:200; step (302) of proceeding a sterilization process to the raw material of the dioscorea and then cooling the product down to a specific temperature, wherein the sterilization process is a high temperature sterilization process, a temperature for the sterilization process is at least 121° C., and a time for the sterilization process is at least 30 minutes; step (303) of inoculating a Monascus spp. to the sterilized dioscorea; step (304) of culturing the inoculated dioscorea under a specific cultural temperature and a specific shaking frequency for a specific cultural time period, wherein the specific cultural temperature is 25˜37° C., the optimal cultural temperature is 30° C., the specific shaking frequency is 50˜300 rpm, the optimal shaking frequency is 200 rpm, the specific cultural time period is 8˜20 days, and the optimal time is 10 days; step (305) of proceeding an anaerobic treatment to the product of previous step for a specific treating time period, wherein the specific treating time period is 3 days, and the optimal time is 2 days; and step (306) of drying the product of previous step to a specific water content so as to accomplish the manufacture of red mold dioscorea, wherein the specific water content is below 15%, and the optimal water content is 6%.

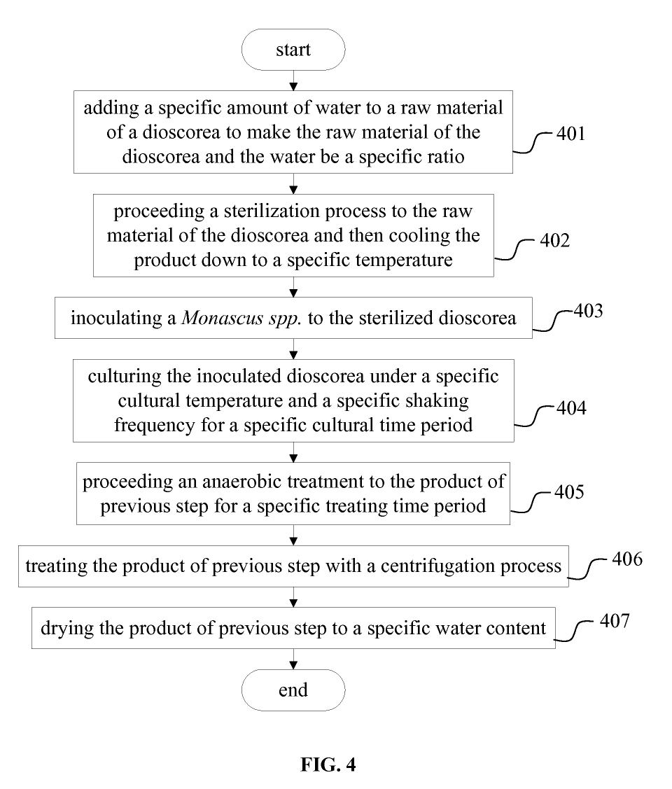

Referring to FIG. 4, which is a flowchart of a method for manufacturing red mold dioscorea according to the fourth preferred embodiment of the present invention. The steps (401)˜(405) and (407) of the fourth preferred embodiment are almost the same as the steps (301)˜(305) and (306) of the third preferred embodiment. The difference is that the product of step (405) of the fourth preferred embodiment can be treated with a centrifugation process of step (406) to obtain a higher purity and then treated with the final drying method of step (407) so as to accomplish the manufacture of red mold dioscorea.

The raw material of the dioscorea in the initial steps of the third and fourth preferred embodiments can be selected from the group consisted of: a whole fresh dioscorea; a fresh dioscorea cut into pieces, wherein a dimension of the pieces is 2˜20 mm; a dried dioscorea, wherein a water content of the dried dioscorea is below 15%; and a dried dioscorea containing sulfur, wherein a water content of the dried dioscorea containing sulfur is below 15%, and a sulfur content is below 160 ppm.

After adding the specific amount of water to the raw material of dioscorea in step (301) and (401) of the foregoing third and fourth preferred embodiment, the ratio of the dioscorea and the water is 1:10˜1:200, it is obvious that a large amount of water is added. The dioscorea is transformed into a liquid form of a dioscorea substrate after being sterilized with high temperature and the dioscorea substrate is treated with a shaking method during the culture step, thus the two methods for culturing red mold yeast with the liquid form of the dioscorea substrate are called liquid state cultivation methods.

In recent years, the secondary metabolites of red mold yeast are paid more and more attention by people, wherein monascin and ankaflavin, the yellow pigments, are proved to be the anti-inflammation agents and the active ingredients for lowering the incidence of cancer, and monacolin K is an effective cholesterol-lowering material, thus promoting the contents of monascin, ankaflavin and monacolin K in the Monascus fermented products is recently an important goal for Monascus researches. In the past, red mold related products are all made from rice as the fermented substrate, but the yields of anti-inflammation material (monascin), anti-cancer material (ankaflavin) and cholesterol-lowering material (monacolin K) are not abundant in red mold rice. In order to increase the amounts of these active ingredients, the present invention use dioscorea as the fermented substrate. The previous literatures do not show that dioscorea can be used as the fermented substrate, but dioscorea is rich in starch and possess a better water retention property, therefore dioscorea is suitable for the growth of red mold yeast.

Red mold dioscorea can be manufactured successfully according to the method of the present invention. There are large differences in the appearances between red mold dioscorea and red mold rice, wherein the largest distinction is that the major pigment produced by red mold rice is red pigment and the appearance of red mol dioscorea is orange-yellow.

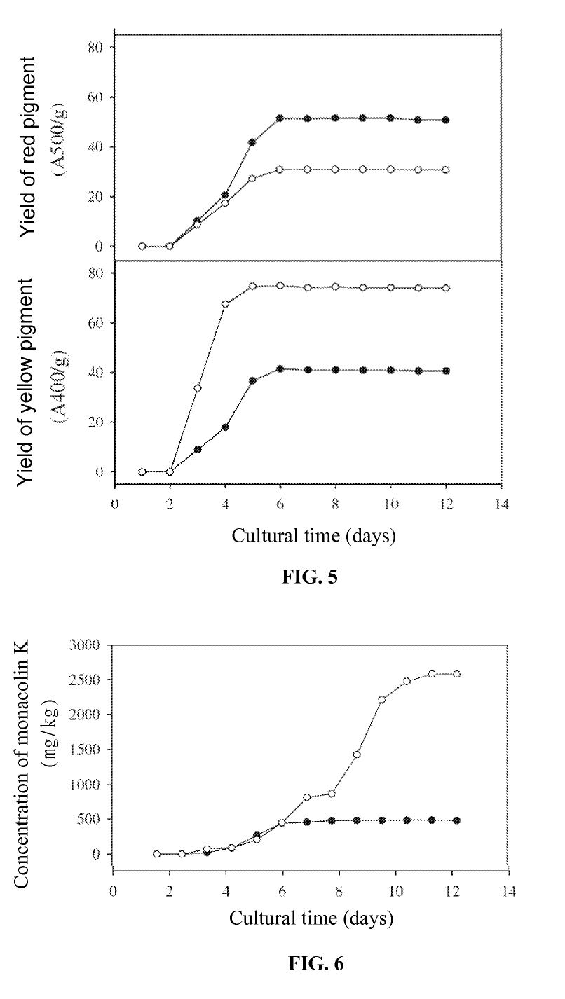

Referring to FIG. 5, which is a trend graph of yellow pigment and red pigment formation in the cultural processes of red mold rice (solid circle) and red mold dioscorea (hollow circle). A large amount of yellow pigment is produced by red mold dioscorea in the initial stage, and the yield of yellow pigment in red mold dioscorea is higher than that in red mold rice; in terms of red pigment, the yield of red pigment in red mold dioscorea is lower than that in red mold rice. These results demonstrate that red mold yeast fermented with dioscorea can produce a large amount of yellow pigment containing some active ingredients, such as monascin and ankaflavin.

Referring to Table 1, which compares the yield of monascin, ankaflavin and GABA in red mold dioscorea and red mold rice cultured with different methods.

TABLE 1

| | Monascin | Ankaflavin | GABA |
|---|---|---|---|
| | (mg/kg) | (mg/kg) | (mg/kg) |
| Red mold rice - | 3547 | 1598 | 131 |
| solid state cultivation | | | |
| Red mold dioscorea - | 15011 | 10074 | 513 |
| solid state cultivation | | | |
| Red mold rice - | 5415 | 2488 | 45 |
| liquid state cultivation | | | |
| Red mold dioscorea - | 23280 | 15330 | 46 |
| liquid state cultivation | | | |

As shown Table 1, the yield of monascin in red mold dioscorea resulted from solid state cultivation is 4.23 times more than that in red mold rice resulted from solid state cultivation, and the yield of ankaflavin in red mold dioscorea resulted from solid state cultivation is elevated by 6.30 times significantly contrasting with red mold rice. This shows that the contents of monascin and ankaflavin in red mold dioscorea cultured by solid state cultivation is higher than that in red mold rice cultured by solid state cultivation. Red mold dioscorea produced by liquid state cultivation has the same effects as above results. The contents of monascin and ankaflavin in red mold dioscorea cultured by liquid state cultivation are 4.30 and 5.16 times respectively more than that in red mold rice cultured by liquid state cultivation. Furthermore, the production quantity of GABA is also higher in the cultural products in red mold dioscorea.

Referring to FIG. 6, which is a trend graph of monacolin K formation in the cultural processes of red mold rice (solid circle) and red mold dioscorea (hollow circle). The trend graph illustrates that the slow production of monacolin K in red mold rice begins at the second day of the cultivation and stagnates at the sixth day; red mold dioscorea produces monacolin K constantly until the tenth day, wherein the production of monacolin K is very rapid from the fifth day to the tenth day. Additionally, after completing the cultivation, the yield of monacolin K in red mold dioscorea is over 5 times more than that in red mold rice.

Referring to Table 2, which compares the yields of monacolin K, red pigment and yellow pigment in several kinds of substrates.

TABLE 2

| | | Red pigment | Yellow pigment |
|---|---|---|---|
| | Monacolin K (mg/kg) | (A500/g) | (A400/g) |
| dioscorea | 2584 ± 127 | 30 ± 5.0 | 74 ± 4.7 |
| rice | 481 ± 33 | 50 ± 4.2 | 41 ± 4.8 |
| cassava | 522 ± 34 | 50 ± 3.8 | 34 ± 3.8 |
| Sweet potato | 196 ± 21 | 51 ± 3.2 | 39 ± 3.9 |
| potato | 495 ± 37 | 48 ± 6.9 | 32 ± 4.8 |

As shown in Table 2, it is obvious that the yield of monacolin K in red mold dioscorea is 5.27 times more than that in red mold rice, 4.95 times more than that in cassava, 13.18 times more than that in sweet potato, and 5.22 times more than that in potato. Thus, dioscorea is the best substrate for producing monacolin K in these substrates. Furthermore, data in Table 2 also indicate that the yield of yellow pigment in red mold dioscorea is higher than that in other substrates, hence red mold dioscorea indeed can promote the yield of yellow pigment, such as monascin and ankaflavin.

From the above researches, the results demonstrate that red mold dioscorea possesses higher yields of monascin, ankaflavin, GABA, and monacolin K, which are active ingredients with the effects of anti-inflammation, anti-cancer, blood pressure lowering, nerve conduction promoting, and cholesterol lowering, and can achieve well effects of blood lipid lowering, blood pressure lowering, atherosclerosis preventing, and Alzheimer's disease improving, thus red mold dioscorea is highly important in the researches and developments of health foods in future. The present invention opens up and develops red mold dioscorea possessing higher yields of functional metabolites comparing with that in red mold rice, and it is expected to be contributive to the developments of Monascus related products.

The foregoing embodiments are provided to illustrate and disclose the technical principles and features of the present invention so as to enable persons skilled in the art to understand the disclosure of the present invention and implement the present invention accordingly, and are not intended to be restrictive of the scope of the present invention. Hence, all equivalent modifications and variations made to the foregoing embodiments without departing from the spirit and principles in the disclosure of the present invention should fall within the scope of the invention as set forth in the appended claims.

What is claimed is:

1. A method for manufacturing red mold dioscorea comprising the steps of:
(1) washing a fresh dioscorea clean and cutting the fresh dioscorea into pieces with a specific dimension;
(2) drying the pieces of the fresh dioscorea to a specific water content;
(3) adding a specific amount of water to the dried dioscorea to make the dried dioscorea and the water be a specific ratio in volum and soaking the dried dioscorea for a specific time period;
(4) proceeding a sterilization process to the soaked dioscorea and then cooling the product down to a specific temperature;
(5) inoculating a Monascus spp. to the sterilized dioscorea;
(6) culturing the inoculated dioscorea under a specific cultural temperature and a specific cultural humidity for a specific cultural time period;
(7) proceeding an anaerobic treatment to the product of step (6) for a specific treating time period; and
(8) drying the product of step (7) to a specific water content.

2. The method for manufacturing red mold dioscorea according to claim 1, wherein the specific dimension in step (1) is 2˜20 mm.

3. The method for manufacturing red mold dioscorea according to claim 1, wherein the specific water content in step (2) is below 15%.

4. The method for manufacturing red mold dioscorea according to claim 1, wherein the specific ratio in step (3) is 1:0.5%˜1:1.5%.

5. The method for manufacturing red mold dioscorea according to claim 1, wherein the specific time period in step (3) is 60 minutes.

6. The method for manufacturing red mold dioscorea according to claim 1, wherein the sterilization process in step (4) is a high temperature sterilization process, a temperature for the sterilization process is at least 121° C., and the specific time period for the sterilization process is 10˜60 minutes.

7. The method for manufacturing red mold dioscorea according to claim 1, wherein the specific cultural temperature in step (6) is 25˜37° C., the specific cultural humidity is 50˜80%, and the specific cultural time period is 8˜20 days.

8. The method for manufacturing red mold dioscorea according to claim 1, wherein the specific treating time period in step (7) is 3 days.

9. The method for manufacturing red mold dioscorea according to claim 1, wherein the specific water content in step (8) is below 15%.

10. The method for manufacturing red mold dioscorea according to claim 1, wherein the pieces of the fresh dioscorea in step (2) can be dried by a sulfuring method to reach a specific sulfur content.

11. The method for manufacturing red mold dioscorea according to claim 10, wherein the specific sulfur content is below 160 ppm.

12. A method for manufacturing red mold dioscorea comprising the steps of:
(1) adding a specific amount of water to a raw material of a dioscorea to make the raw material of the dioscorea and the water be a specific ratio;
(2) proceeding a sterilization process to the raw material of the dioscorea and then cooling the product down to a specific temperature;
(3) inoculating a Monascus spp. to the sterilized dioscorea;
(4) culturing the inoculated dioscorea under a specific cultural temperature and a specific shaking frequency for a specific cultural time period;
(5) proceeding an anaerobic treatment to the product of step (4) for a specific treating time period; and
(6) drying the product of step (5) to a specific water content.

13. The method for manufacturing red mold dioscorea according to claim 12, wherein the raw material of the dioscorea in step (1) is selected from the group consisting of: a whole fresh dioscorea, a fresh dioscorea cut into pieces, a dried dioscorea, and a dried dioscorea containing sulfur.

14. The method for manufacturing red mold dioscorea according to claim 13, wherein a dimension of the fresh dioscorea cut into pieces is 2˜20 mm.

15. The method for manufacturing red mold dioscorea according to claim 13, wherein a water content of the dried dioscorea is below 15%.

16. The method for manufacturing red mold dioscorea according to claim 13, wherein a water content of the dried dioscorea containing sulfur is below 15%, and a sulfur content is below 160 ppm.

17. The method for manufacturing red mold dioscorea according to claim 12, wherein the specific ratio in step (1) is 1:10˜1:200.

18. The method for manufacturing red mold dioscorea according to claim 12, wherein the sterilization process in step (2) is a high temperature sterilization process, a temperature for the sterilization process is at least 121° C., and a time for the sterilization process is at least 30 minutes.

19. The method for manufacturing red mold dioscorea according to claim 12, wherein the specific cultural temperature in step (4) is 25˜37° C., the specific shaking frequency is 50˜300 rpm, and the specific cultural time period is 8˜20 days.

20. The method for manufacturing red mold dioscorea according to claim 12, wherein the specific treating time period in step (5) is 3 days.

21. The method for manufacturing red mold dioscorea according to claim 12, wherein the specific water content in step (6) is below 15%.

22. The method for manufacturing red mold dioscorea according to claim 12, wherein the product of step (5) can be treated with a centrifugation process before going to step (6).
